(19) United States
(12) Patent Application Publication
(10) Pub. No.: US 2013/0045874 A1
(43) Pub. Date: Feb. 21, 2013

(54) Method of Diagnostic of Inflammatory Bowel Diseases

(71) Applicant: Stanislav Ehrlich, Orsay (FR)

(72) Inventor: Stanislav Ehrlich, Orsay (FR)

(73) Assignee: INSTITUT NATIONAL DE LA RECHERCHE AGRONOMIQUE, Paris (FR)

(21) Appl. No.: 13/582,405

(22) PCT Filed: Mar. 1, 2011
(86) PCT No.: PCT/EP2011/053039

§ 371 Date: Aug. 31, 2012

Related U.S. Application Data
(60) Provisional application No. 61/309,302, filed on Mar. 1, 2010.

(51) Int. Cl. C12Q 1/68; C40B 40/06; C40B 20/00

(52) U.S. Cl. 506/2; 435/6.12; 435/6.11; 506/16

(57) ABSTRACT

A new method for diagnosing an inflammatory bowel disease is herein described, based on the determination of the absence of at least one gene from the human' gut microbiome.

### CROSS-REFERENCE TO RELATED APPLICATIONS

This application is a U.S. National Stage application under 35 U.S.C. 371 of PCT/EP2011/053039, filed Mar. 1, 2011, which claims the benefit of U.S. provisional application 61/309,302, filed Mar. 1, 2010. Each of these applications is incorporated by reference in its entirety herein.

Inflammatory bowel diseases are chronic disorders of unknown aetiology characterized by persistent mucosal inflammation at different levels of the gastrointestinal tract. Ulcerative colitis and Crohn's disease are the two main types of inflammatory bowel diseases. Ulcerative colitis causes continuous mucosal inflammation that is restricted to the colon whereas Crohn's disease causes discontinuous transmural inflammation anywhere throughout the gastrointestinal tract, although it most frequently affects the terminal ileum. Most common intestinal lesions consist of mucosal ulcerations, bowel wall swelling and stricturing of the intestinal lumen. These chronic inflammatory lesions may cause symptoms such as diarrhoea, faecal urgency, abdominal pain and fever, as well as complications of variable severity including bleeding, intestinal obstruction, sepsis and malnutrition.

Epidemiological studies demonstrated a steady increase of the incidence of such diseases in Western Europe and North America during the last century since 1950. In Southern Europe and Japan the rise in incidence came two decades later, but today incidence rates are as important as in Northern Europe and North America. Recent data suggest increasing incidence in Eastern European countries as well as in South and East Asia. Changes in incidence seem to be related to the westernization of lifestyles, including changes in dietary habits and environmental changes such as improved sanitation and industrialization. Today, figures of combined prevalence (ulcerative colitis plus Crohn's disease) suggest that inflammatory bowel diseases affects up to 0.5% of the population of developed societies.

The impact of inflammatory bowel diseases on society is disproportionately high, as presentation often occurs at a young age and has the potential to cause lifelong ill health. At present, there is no cure or eradication therapy for inflammatory bowel diseases. Typically, both ulcerative colitis and Crohn's disease exhibit undulating activity with bouts of uncontrolled, chronic mucosal inflammation, followed by remodelling processes that occur during periods of remission. The primary treatment approach is usually drug therapy to mitigate bouts of inflammatory activity and to prevent future relapses when in remission. Patients can be treated with a variety of drugs, including 5-ASAs (e.g. mesalazine), steroids (e.g. prednisolone) and immunosuppressants (e.g. azathioprine). In addition, patients may also receive new biological drugs such as monoclonal antibodies (e.g. the anti-TNF-α antibody infliximab) when standard drug treatment fails. Despite their general efficacy, such drugs can carry a significant burden. They are not only expensive, but side effects are common, with an incidence of 28% for immunosuppressants, rising to 50% for steroids. Some patients may present severe side effects like systemic infections or neoplasia, and therefore current therapies require a close surveillance. In addition, approximately 30% of patients with ulcerative colitis and 50% of patients with Crohn's disease will require surgery at some point in their life.

Available medical therapies cannot achieve eradication or permanent cure of such diseases, and this is mainly due to that fact that the precise aetiologies of ulcerative colitis and Crohn's disease remain to be elucidated. However, the pathophysiological mechanisms that lead to the mucosal inflammatory lesions have been unveiled at least in part during the past few years. There is convincing evidence that the inflammation observed in inflammatory bowel diseases is caused by abnormal communication between the gut microbial communities and the mucosal immune system. The defensive response of T helper lymphocytes (Th) of the mucosal immune system against pathogens is associated with inflammatory processes aimed at the eliminations of the pathogen, but at the same time these processes also damage the host tissues. Under normal circumstances, some commensal gut microbes seem to play a major role for induction of regulatory T lymphocytes (Tregs) in gut lymphoid follicles. Regulatory T lymphocytes are key players of the phenomenon called ‘immune tolerance’, since these lymphocytes do not induce inflammation in response to the microbial antigens that are recognised as non-pathogenic. Immune tolerance mediated by regulatory T lymphocytes is the essential homeostatic mechanism by which the host can tolerate the massive burden of innocuous antigens within the gut or on other body surfaces without responding through inflammation. Several lines of evidence suggest that in individuals with genetic susceptibility, Th lymphocyte-mediated immunity against luminal bacteria is the key event in driving the inflammatory process that generates intestinal lesions and/or impairs resolution of the lesions. A defective interaction of the gut microbiota with the mucosal immune compartments may result in the abnormalities leading to chronic intestinal inflammation.

Several studies have shown that the composition of the faecal microbiota differs between subjects with inflammatory bowel diseases and healthy controls. The reported differences are variable and not always consistent among the various studies. It is thus not possible to use the published differences to distinguish between patients with inflammatory bowel diseases and healthy people. However, as explained above, the indigenous gut microbes will be determinant under certain circumstances in the onset and maintenance of inflammatory bowel diseases, especially Crohn's disease. There is thus still a need for a new, reliable method allowing a consistent diagnostic of inflammatory bowel diseases.

Most intestinal commensals cannot be cultured. Genomic strategies have been developed to overcome this limitation (Hamady and Knight, Genome Res, 19: 1141-1152, 2009). These strategies have allowed the definition of the microbiome as the collection of the genes comprised in the genomes of the microbiota (Turnbaugh et al., Nature, 449: 804-8010, 2007; Hamady and Knight, Genome Res., 19: 1141-1152, 2009). The existence of a small number of species shared by all individuals constituting the human intestinal microbiota phylogenetic core has been demonstrated (Tap et al., Environ Microbiol., 11(10): 2574-2584, 2009). Recently, a metagenomic analysis has led to the identification of an extensive catalogue of 3.3 million non-redundant microbial genes of the human gut, corresponding to 576.7 gigabases of sequence (Qin et al., Nature, 2010, doi:10.1038/nature08821).

The inventors have used a method based on the isolation and sequencing of DNA fragments from human faeces in different individuals. Since an extensive catalogue of microbial genes from the gut is now available (Qin et al., Nature, 2010, doi:10.1038/nature08821), the number of copies and the frequency of a specific sequence in a specific population (e.g. patients suffering from inflammatory bowel diseases) can be calculated. It is thus possible to identify any correlation between the presence or absence of a specific gene and the presence or absence of a specific pathology. In addition, the number of copies of a specific gene in an individual can be determined.

Crohn's disease and ulcerative colitis are chronic immune inflammatory conditions of the alimentary tract, referred to collectively as inflammatory bowel diseases. The inventors were able to identify genes which are significantly different between a group of patient suffering from Crohn's disease or ulcerative colitis, and a control group of healthy people. These genes are listed in Table 1 (Crohn's diseaese) and Table 2 (ulcerative colitis). The said genes are more numerous in healthy individuals than in the patients. This observation is statistically significant, since the total number of microbial genes is not different in both populations. There is thus a loss of specific human's gut microbial genes in individuals suffering from inflammatory bowel disease.

A first aspect of this invention is a method for diagnosing an inflammatory bowel disease, said method comprising a step of determining whether at least one gene is absent from an individual's gut microbiome. By “individual's gut microbiome”, it is herein understood all the genes constituting the microbiota of the said individual. The term “individual's gut microbiome” thus corresponds to all the genes of all the bacteria present in the said individual's gut.

A gene is absent from the microbiome when its number of copies in the microbiome is under a certain threshold value. According to the present invention, a “threshold value” is intended to mean a value that permits to discriminate samples in which the number of copies of the gene of interest corresponds to a number of copies in the individual's microbiome that is low or high. In particular, if a number of copies is inferior or equal to the threshold value, then the number of copies of this gene in the microbiome is considered low, whereas if the number of copies is superior to the threshold value, then the number of copies of this gene in the microbiome is considered high. A low copy number means that the gene is absent from the microbiome, whereas a high number of copies means that the gene is present in the microbiome. For each gene, and depending on the method used for measuring the number of copies of the gene, the optimal threshold value may vary. However, it may be easily determined by a skilled artisan based on the analysis of the microbiome of several individuals in which the number of copies1 (low or high) is known for this particular gene, and on the comparison thereof with the number of copies of a control gene.

The method of the invention thus allows the skilled person to diagnose a pathology solely on the basis of the presence or the absence of a gene from the individual's gut microbiome. There is a direct correlation between the number of copies of a specific gene and the number of bacterial cells carrying this gene. The method of the invention thus allows the skilled person to detect a dysbiosis, i.e. a microbial imbalance, by analysis of the microbiome. Not all the species in the gut have been identified, because most cannot be cultured, and identification is difficult. In addition, most species found in the gut of a given individual are rare, which makes them difficult to detect (Hamady and Knight, Genome Res., 19: 1141-1152, 2009). In this first aspect of the invention, no prior identification of the bacterial species the said gene belongs to is required. The method of diagnosis of the invention is thus not restricted to the determination of a change in the population of known gut's bacterial species, but encompasses also the bacteria which have not yet been characterized taxiconomically.

There are several ways to obtain samples of the said individual's gut microbial DNA (Sokol et al., Inflamm. Bowel Dis., 14(6): 858-867, 2008). For example, it is possible to prepare mucosal specimens, or biopsies, obtained by colonoscopy. However, colonoscopy is an invasive procedure which is ill-defined in terms of collection procedure from study to study. Likewise, it is possible to obtain biopsies through surgery. However, even more than colonoscopy, surgery is an invasive procedure, which effects on the microbial population are not known. Preferred is the faecal analysis, a procedure which has been reliably been used in the art (Bullock et al., Curr Issues Intest Microbiol.; 5(2): 59-64, 2004; Manichanh et al., Gut, 55: 205-211, 2006; Bakir et al., Int J Syst Evol Microbiol, 56(5): 931-935, 2006; Manichanh et al., Nucl. Acids Res., 36(16): 5180-5188, 2008; Sokol et al., Inflamm. Bowel Dis., 14(6): 858-867, 2008). An example of this procedure is described in the Methods section of the Experimental Examples.

Faeces contain about 10¹¹ bacterial cells per gram (wet weight) and bacterial cells comprise about 50% of faecal mass. The microbiota of the faeces represent primarily the microbiology of the distal large bowel. It is thus possible to isolate and analyse large quantities of microbial DNA from the faeces of an individual. By “microbial DNA”, it is herein understood the DNA from any of the resident bacterial communities of the human gut. The term “microbial DNA” encompasses both coding and non-coding sequences; it is in particular not restricted to complete genes, but also comprises fragments of coding sequences. Faecal analysis is thus a non-invasive procedure, which yields consistent and directly comparable results from patient to patient.

Therefore, in a preferred embodiment, the method of the invention comprises a step of obtaining microbial DNA from faeces of the said individual. In a further preferred embodiment, the faeces from said individual are collected, DNA is extracted, and the presence or absence from an individual's gut microbiome of at least one gene is determined. The presence or absence of a gene may be determined by all the methods known to the skilled person. For instance, the whole microbiome of the said individual may be sequenced, and the presence or absence of the said gene searched with the help of bioinformatics methods. One instance of such a strategy is described in the Methods section of the Experimental Examples.

Alternatively, the gene of interest may be looked for in the microbiome by hybridization with a specific probe, e.g. by Southern hybridization. It will be immediately apparent to the person of skills in the art that, in this particular embodiment, although Southern hybridization is perfectly suitable, it is nevertheless more convenient and sensitive to use microarrays. In yet another embodiment, the presence of the gene of interest may be detected by amplification, in particular by quantitative PCR (qPCR). These technologies (Southern, microarrays, qPCR, etc) are now used routinely by those skilled in the art and thus do not need to be detailed here.

In another preferred embodiment, the inflammatory bowel disease is selected from the group of Crohn's disease and ulcerous colitis. In a further preferred embodiment, the said disease is Crohn's disease; in another further preferred embodiment, the said disease is ulcerous colitis.

In yet another preferred embodiment, the gene which absence or presence from the individual's gut microbiome is determined is selected from the group of genes listed in Tables 1 and 2. In a further preferred embodiment, the gene is selected from the group of genes listed in Table 1; in another further preferred embodiment, the gene is selected from the group of genes listed in Table 2. The skilled person will have no difficulty in realizing that the more genes are tested, the higher the degree of confidence of the result. According to another further preferred embodiment, the method of the invention comprises determining the presence or absence of at least 50% of the genes listed in Table 1, more preferably, at least 75% of the genes of Table 1, even more preferably, at least 90% of the genes of Table 1. According to another further preferred embodiment, the method of the invention comprises determining the presence or absence of at least 50% of the genes listed in Table 2, more preferably, at least 75% of the genes of Table 2, even more preferably, at least 90% of the genes of Table 2.

Even though a great number of the bacterial species found in the microbial flora have not been identified, it is known that most bacteria belong to the genera Bacteroides, Clostridium, Fusobacterium, Eubacterium, Ruminococcus, Peptococcus, Peptostreptococcus, and Bifidobacterium. Other genera such as Escherichia and Lactobacillus are present to a lesser extent. Some individual species belonging to these genera have been identified, and some of the genes of these species are known. The extensive metagenomic study which has led to the identification of 3.3 million non-redundant microbial genes has also permitted the assignment of most new sequences. A gene belonging to a given species is present in an individual at the same frequency as all the other genes of the said species. It is thus possible for each of the genes identified through the method of the invention to determine whether there is a correlation between the presence or absence of the said gene and the presence or absence of a set of genes known to belong to a specific bacterial species in various individuals. Such a correlation indicates that the unknown gene belongs to the said specific bacterial species. The inventors have thus shown that some bacterial species are associated with the inflammatory bowel disease phenotype whereas other bacterial species are associated with the healthy phenotype. The inflammatory bowel disease phenotype can be predicted by a linear combination of the said species, i.e. the more bacterial species associated with the inflammatory bowel disease phenotype are present in an individual's gut, and the lesser species associated with the healthy phenotype in the said individual's gut, the higher the probability that the said individual suffers from an inflammatory disease. For example, the absence of Faecalibacterium prausnitzii and Roseburia inulinivorans and the presence of Clostridium boltae, Clostridium ramosum and Ruminococcus gnavus in the gut of a person indicates that this person suffers from Crohn's disease. Likewise, the absence of Akkermansia muciniphila and the presence of Bacteroides capillosus and Clostridium leptum in an individual's gut indicates that this person suffers from ulcerative colitis.

It will be clear for the person skilled in the art that the genes of the invention can be used as biomarkers, for example during the treatment of patients suffering from inflammatory bowel diseases. Therefore, in another embodiment, the invention includes a method for monitoring the efficacy of a treatment for an inflammatory bowel disease. When a treatment is efficacious against an inflammatory bowel disease, the dysbiosis initially observed gradually disappears. Whereas some specific genes are absent from the individual's guts when that said individual is sick (e.g. the genes of Table 1 when the disease is Crohn's disease, or the genes of Table 2, when the individual suffers from ulcerous colitis), these genes reappear during the treatment. In this embodiment, the method of the invention thus comprises the steps of first determining whether at least one gene is absent from the said patient's microbiome, administering the treatment, determining if the said at least one gene is present in the patient's microbiome. In a preferred embodiment, the method of the invention comprises the steps of obtaining microbial DNA from faeces of the said individual, before and after the treatment. In a further preferred embodiment, the faeces from said individual are collected before and after the treatment, DNA is extracted, and the presence or absence from an individual's gut microbiome of at least one gene is determined.

In another preferred embodiment, the inflammatory bowel disease is selected from the group of Crohn's disease and ulcerous colitis. In a further preferred embodiment, the said disease is Crohn's disease; in another further preferred embodiment, the said disease is ulcerous colitis.

In yet another preferred embodiment, the gene which absence or presence from the individual's gut microbiome is determined is selected from the group of genes listed in Tables 1 and 2. In a further preferred embodiment, the gene is selected from the group of genes listed in Table 1; in another further preferred embodiment, the gene is selected from the group of genes listed in Table 2. In a particular embodiment of the method of the invention, at least 50%, 75% or 90% of the genes of Table 1 and/or Table 2 are absent from the said individual's gut microbiome before the treatment. Therefore, according to a preferred embodiment, the method of the invention comprises determining the presence or absence of at least 50% of the genes listed in Table 1, more preferably, at least 75% of the genes of Table 1, even more preferably, at least 90% of the genes of Table 1. According to another preferred embodiment, the method of the invention comprises determining the presence or absence of at least 50% of the genes listed in Table 2, more preferably, at least 75% of the genes of Table 2, even more preferably, at least 90% of the genes of Table 2.

The present invention also includes a kit dedicated to the implementation of the methods of the invention, comprising all the genes which are absent in a patient suffering from an inflammatory bowel disease and which are present in a healthy person. In particular, the present invention relates to a microarray dedicated to the implementation of the methods according to the invention, comprising probes binding to all the genes absent in a patient suffering from an inflammatory bowel disease and present in a healthy person. In a preferred embodiment, said microarray is a nucleic acid microarray. According to the invention, a “nucleic microarray” consists of different nucleic acid probes that are attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes can be nucleic acids such as cDNAs (“cDNA microarray”) or oligonucleotides (“oligonucleotide microarray”, the oligonucleotides being about 25 to about 60 base pairs or less in length). Alternatively to nucleic acid technology, quantitative PCR may be used and amplification primers specific for the genes to be tested are thus also very useful for performing the methods according to the invention. The present invention thus further relates to a kit for diagnosing an inflammatory bowel disease in a patient, comprising a dedicated microarray as described above or amplification primers specific for genes absent in a patient suffering from an inflammatory bowel disease and present in a healthy person. Whereas these kits may allow the skilled person to detect 10%, 25%, 50% or 75% of the said genes, they are most useful when they allow the detection of 90%, 95%, 97.5% or even 99% of the said genes. Thus a microarray according to the invention will comprise probes binding to at least 10%, 25%, 50% or 75%, and preferably 90%, 95%, 97.5%, and even more preferably at least 99% of the said genes. Likewise a kit for quantitative PCR will contain primers allowing the amplification of at least 10%, 25%, 50% or 75%, and preferably 90%, 95%, 97.5%, and even more preferably at least 99% of the said genes.

In a preferred embodiment, the inflammatory bowel disease is selected from the group of Crohn's disease and ulcerous colitis. In a further preferred embodiment, the said disease is Crohn's disease; in another further preferred embodiment, the said disease is ulcerous colitis. In another embodiment, the genes which are absent in a patient suffering from Cohn's disease and are present in healthy people are the genes listed in Table 1; in yet another embodiment, they are listed in Table 2.

### FIGURE LEGENDS

FIG. 1: Overall analysis of the CD-related genes and of UC-related genes. A) More CD-related genes in healthy individuals. Plot of the number of genes per individual in function the CD-related genes indicates that the genes are more numerous in healthy individuals than the patients. B) More UC-related genes in healthy individuals. Plot of the number of genes per individual in function the UC-related genes indicates that the genes are more numerous in healthy individuals than the patients.

FIG. 2: A) A linear combination of the 5 species discriminates well the Crohn's disease phenotype for the part of the cohort that harbors them at the levels defined (at least 50% of the genes); B) 3 species discriminate for ulcerative colitis.

### METHODS

Human faecal sample collection. Spanish individuals were either healthy controls or patients with chronic inflammatory bowel diseases (Crohn's disease or ulcerative colitis) in clinical remission. Patients and healthy controls were asked to provide a frozen stool sample.

Fresh stool samples were obtained at home, and samples were immediately frozen by storing them in their home freezer. Frozen samples were delivered to the hospital using insulating polystyrene foam containers, and then they were stored at −80° C. until analysis.

DNA extraction. A frozen aliquot (200 mg) of each faecal sample was suspended in 250 μl of guanidine thiocyanate, 0.1M Tris (pH 7.5) and 40 μl of 10% N-lauroyl sarcosine. Then, DNA extraction was conducted as previously described (Manichanh et al.,. Gut, 55: 205-211, 2006). The DNA concentration and its molecular size were estimated by nanodrop (Thermo Scientific) and agarose gel electrophoresis.

DNA library construction and sequencing. DNA library preparation followed the manufacturer's instruction (Illumina). We used the same workflow as described elsewhere to perform cluster generation, template hybridization, isothermal amplification, linearization, blocking and denaturization and hybridization of the sequencing primers. The base-calling pipeline (version IlluminaPipeline-0.3) was used to process the raw fluorescent images and call sequences. We constructed one library (clone insert size 200 bp) for each of the first 15 samples, and two libraries with different clone insert sizes (135 by and 400 bp) for each of the remaining 109 samples for validation of experimental reproducibility. To estimate the optimal return between the generation of novel sequence and sequencing depth, we aligned the Illumina GA reads from samples MH0006 and MH0012 onto 468,335 Sanger reads totaling to 311.7 Mb generated from the same two samples (156.9 and 154.7 Mb, respectively), using the Short Oligonucleotide Alignment Program (SOAP) (Li et al., Bioinformatics, 25: 1966-1967, 2009). and a match requirement of 95% sequence identity. With about 4 Gb of Illumina sequence, 94% and 89% of the Sanger reads (for MH0006 and MH0012, respectively) were covered. Further extensive sequencing, to 12.6 and 16.6 Gb for MH0006 and MH0012, respectively, brought only a moderate increase of coverage to about 95%. More than 90% of the Sanger reads were covered by the Illumina sequences to a very high and uniform level, indicating that there is little or no bias in the Illumina GA sequence. As expected, a large proportion of Illumina sequences (57% and 74% for M0006 and M0012, respectively) was novel and could not be mapped onto the Sanger reads. This fraction was similar at the 4 and 12-16 Gb sequencing levels, confirming that most of the novelty was captured already at 4 Gb.

We generated 35.4-97.6 million reads for the remaining 122 samples, with an average of 62.5 million reads. Sequencing read length of the first batch of 15 samples was 44 by and the second batch was 75 bp.

Public data used The sequenced bacteria genomes (totally 806 genomes) deposited in GenBank were downloaded from the NCBI database (http://www.ncbi.nlm.nih.gov/) on 10 Jan. 2009. The known human gut bacteria genome sequences were downloaded from HMP database (http://www.hmpdacc-resources.org/cgi-bin/hmp_catalog/main.cgi), GenBank (67 genomes), Washington University in St Louis (85 genomes, version April 2009, http://genome.wustl.edu/pub/organism/Microbes/Human_Gut Microbiome/), and sequenced by the MetaHIT project (17 genomes, version September 2009, http://www.sanger.ac.uk/pathogens/metahit/). The other gut metagenome data used in this project include: (1) human gut metagenomic data sequenced from US individuals (Zhang et al., Proc. Natl Acad. Sci. USA, 106: 2365-2370, 2009), which was downloaded from NCBI with the accession SRA002775; (2) human gut metagenomic data from Japanese individuals (Kurokawa et al., DNA Res. 14: 169-181, 2007), which was downloaded from P. Bork's group at EMBL (http://www.bork.embl.de). The integrated NR database we constructed in this study included NCBI-NR database (version April 2009) and all genes from the known human gut bacteria genomes.

Illumina GA short reads de novo assembly. High-quality short reads of each DNA sample were assembled by the SOAP de novo assembler (Li. & Zhu, Genome Res., 20(2): 265-272, 2010). In brief, we first filtered the low abundant sequences from the assembly according to 17-mer frequencies The 17-mers with depth less than 5 were screened in front of assembly, for these low-frequency sequences were very unlikely to be assembled, whereas removing them would significantly reduce memory requirement and make assembly feasible in an ordinary supercomputer (512 GB memory in our institute). Then the sequences were processed one by one and the de Bruijn graph data format was used to store the overlap information among the sequences. The overlap paths supported by a single read were unreliable and removed. Short low-depth tips and bubbles that were caused by sequencing errors or genetic variations between microbial strains were trimmed and merged, respectively. Read paths were used to solve the tiny repeats. Finally, we broke the connections at repeat boundaries, and outputted the continuous sequences with unambiguous connections as contigs. The metagenomic special model was chosen, and parameters ‘-K 21’ and ‘-K 23’ were used for 44 by and 75 by reads, respectively, to indicate the minimal sequence overlap required. After de novo assembly for each sample independently, we merged all the unassembled reads together and performed assembly for them, as to maximize the usage of data and assemble the microbial genomes that have low frequency in each read set, but have sufficient sequence depth for assembly by putting the data of all samples together.

Validating Illumina contigs using Sanger reads. We used BLASTN (WUBLAST 2.0) to map Sanger reads from samples MH0006 and MH0012 (156.9 Mb and 154.7 Mb, respectively) to Illumina contigs (single best hit longer than 75 by and over 95% identity) from the same samples. Each alignment was scanned for breakage of collinearity where both sequences have at least 50 bases left unaligned at one end of the alignment. Each such breakage was considered an assembly error in the Illumina contig at the location where collinearity breaks. Errors within 30 by from each other were merged. An error was discarded if there exists a Sanger read that agrees with the contig structure for 60 by on both sides of the error. For comparison, we repeated this on a Newbler2 assembly of 454 Titanium reads from MH0006 (550 Mb reads). We estimate 14.12 errors per Mb of contigs for the Illumina assembly, which is comparable to that of the 454 assembly (20.73 per Mb). 98.7% of Illumina contigs that map at least one Sanger read were collinear over 99.55% of the mapped regions, which is comparable to 97.86% of such 454 contigs being collinear over 99.48% of the mapped regions.

Evaluation of human gut microbiome coverage. The Illumina GA reads were aligned against the assembled contigs and known bacteria genomes using SOAP by allowing at most two mismatches in the first 35-bp region and 90% identity over the read sequence. The Roche/454 and Sanger sequencing reads were aligned against the same reference using BLASTN with 1×10⁻⁸, over 100 by alignment length and minimal 90% identity cutoff. Two mismatches were allowed and identity was set 95% over the read sequence when aligned to the GA reads of MH0006 and MH0012 to Sanger reads from the same samples by SOAP.

Gene prediction and construction of the non-redundant gene set. We use MetaGene (Noguchi et al., Nucleic Acids Res., 34, 5623-5630, 2006)—which uses di-codon frequencies estimated by the GC content of a given sequence, and predicts a whole range of ORFs based on the anonymous genomic sequences—to find ORFs from the contigs of each of the 124 samples as well as the contigs from the merged assembly. The predicted ORFs were then aligned to each other using BLAT (Kent et al., Genome Res., 12: 656-664, 2002). A pair of genes with greater than 95% identity and aligned length covered over 90% of the shorter gene was grouped together. The groups sharing genes were then merged, and the longest ORF in each merged group was used to represent the group, and the other members of the group were taken as redundancy. Therefore, we organized the non-redundant gene set from all the predicted genes by excluding the redundancy. Finally, the ORFs with length less than 100 by were filtered. We translated the ORFs into protein sequences using the NCBI Genetic Codes (Ley et al., Nature Rev. Microbiol., 6: 776-788, 2008).

Identification of genes. To make a balance between identifying low-abundance genes and reducing the error-rate of identification, we explored the impact of the threshold set for read coverage required to identify a gene in individual microbiomes. The number of genes decreased about twice when the number of reads required for identification was increased from 2 to 6, and changed slowly thereafter. Nevertheless, to include the rare genes into the analysis, we selected the threshold of 2 reads.

Gene taxonomic assignment. Taxonomic assignment of predicted genes was carried out using BLASTP alignment against the integrated NR database. BLASTP alignment hits with e-values larger than 1×10⁻⁵ were filtered, and for each gene the significant matches which were defined by e-values≦10×e-value of the top hit were retained to distinguish taxonomic groups. Then we determined the taxonomical level of each gene by the lowest common ancestor (LCA)-based algorithm that was implemented in MEGAN (Huson et al., Genome Res., 17: 377-386, 2007). The LCA-based algorithm assigns genes to taxa in the way that the taxonomical level of the assigned taxon reflects the level of conservation of the gene. For example, if a gene was conserved in many species, it was assigned to the LCA rather than to a species.

Gene functional classification. We used BLASTP to search the protein sequences of the predicted genes in the eggNOG database (Jensen et al., Nucleic Acids Res., 36: D250-D254, 2008) and KEGG database (Kanehisa et al., Nucleic Acids Res., 32: D277-D280, 2004) with e-value≦1×10⁻⁵. The genes were annotated as the function of the NOGs or KEGG homologues with lowest e-value. The eggNOG database is an integration of the COG and KOG databases. The genes annotated by COG were classified into the 25 COG categories, and genes that were annotated by KEGG were assigned into KEGG pathways.

Determination of minimal gut bacterial genome. The number of non-redundant genes assigned to the eggNOG clusters was normalized by gene length and cluster copy number. The clusters were ranked by normalized gene number and the range that included the clusters encoding essential Bacillus subtilis genes was determined, computing the proportion of these clusters among the successive groups of 100 clusters. Analysis of the range gene clusters involved, besides iPath projections, use of KEGG and manual verification of the completeness of the pathways and protein machineries they encode.

Determination of total functional complement and minimal metagenome. We computed the total and shared number of orthologous groups and/or gene families present in random combinations of n individuals (with n=52 to 124, 100 replicates per bin). This analysis was performed on three groups of gene clusters: (1) known eggNOG orthologous groups (that is, those with functional annotation, excluding those in which the terms [Uu]ncharacteri[sz]ed, [Uu]nknown, [Pp]redicted or[Pp]utative occurred); (2) all eggNOG orthologous groups; (3) all orthologous groups plus gene families constructed from remaining genes not assigned to the two above categories. Families were clustered from all-against-all BLASTP results using MCL (van Dongen, Ph. D. Thesis, Univ. Utrecht, 2000) with an inflation factor of 1.1 and a bit-score cutoff of 60.

Rarefaction analysis. Estimation of total gene richness was done using EstimateS on 100 randomly picked samples due to memory limitations. Because the CV value was >0.5, both chao2 (classic) and ICE richness estimators were calculated and the larger estimate of the two (ICE) was used. The estimate for this sample size was 3,621,646 genes (ICE) whereas S_{obs} (Mao Tau) was 3,090,575 genes, or 85.3%. The ICE estimator curve did not completely saturate, indicating that additional samples will need to be added to achieve a final, conclusive estimate.

Common bacterial core. To eliminate the influence of very similar strains and assess the presence of known microbial species among the individuals of the cohort, we used 650 sequenced bacterial and archaeal genomes as a reference set. The set was composed from 932 publicly available genomes, which were grouped by similarity, using a 90% identity cutoff and the similarity over at least 80% of the length. From each group only the largest genome was used. Illumina reads from 124 individuals were mapped to the set, for species profiling analysis and the genomes originating from the same species (by differing in size >20%) curated by manual inspection and by using the 16S-based clustering when the sequences were available.

Relative abundance of microbial genomes among individuals. We computed the genome coverage by uniquely mapping Illumina reads and normalized it to 1 Gb of sequence, to correct for different sequencing levels in different individuals. The coverage was summed over all species of the non-redundant bacterial genome set for each individual and the proportion of each species relative to the sum calculated.

Species co-existence network. For the 155 species that had genome coverage by the Illumina reads ≧1% in at least one individual we calculated the pair-wise inter-species Pearson correlations between sequencing depths (abundance) throughout the entire cohort of 124 individuals. From the resulting 11,175 inter-species correlations, correlations less than −0.4 or above 0.4 (n=342) were visualized in a graph using Cytoscape (Shannon et al.,. Genome Res. 13: 2498-2504, 2003). displaying the average genome coverage of each species as node size in the graph.

### RESULTS

A summary description of the cohort & the method used. For Crohn's disease, the size of the cohorts was 8 patients and 13 healthy controls; for ulcerative colitis, it was 12 patients and 12 healthy controls. For each disease, the entire gene catalog of 3.3 million genes was searched by ranksum search for those that are significantly different between the two groups. Gene frequency was normalized by the gene size (larger genes are bigger targets and are seen more often) and the difference in the sequencing extent for different individuals. The number of significantly different genes is affected by the thresholds and the splits into groups. In brief, 3802 “CD (Crohn disease)-related genes” were found at p<3×10⁻⁴ and 4841 “UC (ulcerative colitis)-related genes” were found at p<10⁻³.

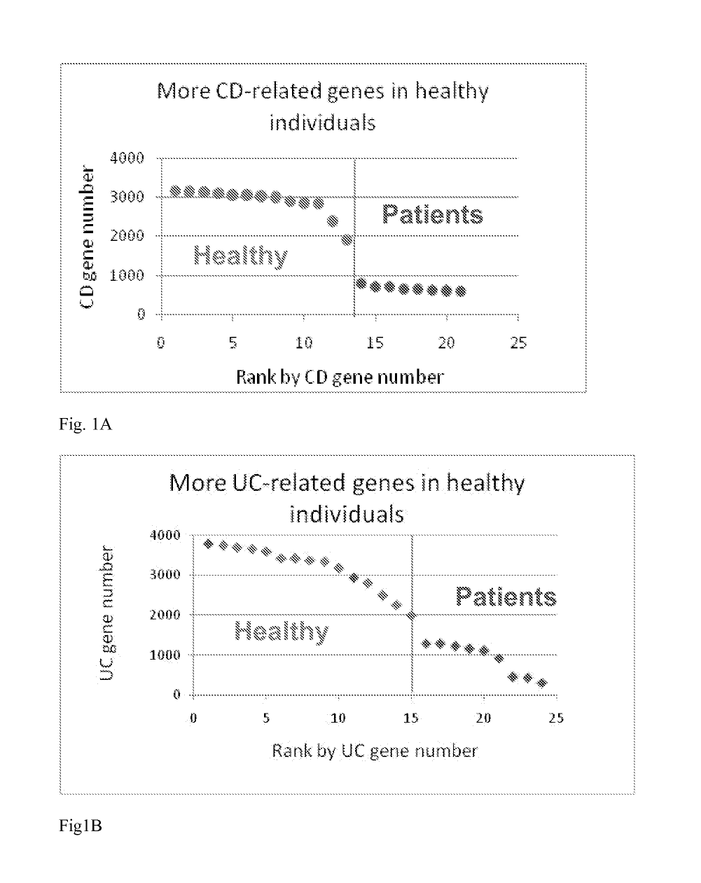

Overall analysis of the BMI genes. The significantly different genes, i.e. either CD-related genes (FIG. 1A) or UC-related genes (FIG. 1B), were plotted by individual. The median number of CD-related genes in a healthy individual was 3038, and only 643 in a Crohn's disease patient. The median gene number is very significantly different among the 2 groups (p<2×10⁻¹³, one-tailed t test). Likewise, the median number of UC-related genes was 3402 in a healthy individual and 1212 in a patient suffering from ulcerative colitis. The difference is statistically different (p<6.7×10⁻⁵, one-tailed t test).

Comparison of the distribution of all genes and CD-related genes or UC-related genes. The distribution of all genes of the microbiome and of the CD-related genes or UC-related genes was compared. There is much less difference in all gene numbers and frequency between the two groups than the CD-related genes or UC-related genes. The CD-related gene distribution does not reflect simply the all gene distribution; similarly, the UC-related gene distribution does not simply reflect a general trend in gene distribution. The loss of genes in the Crohn's disease patients and in the ulcerative disease patients is thus significant.

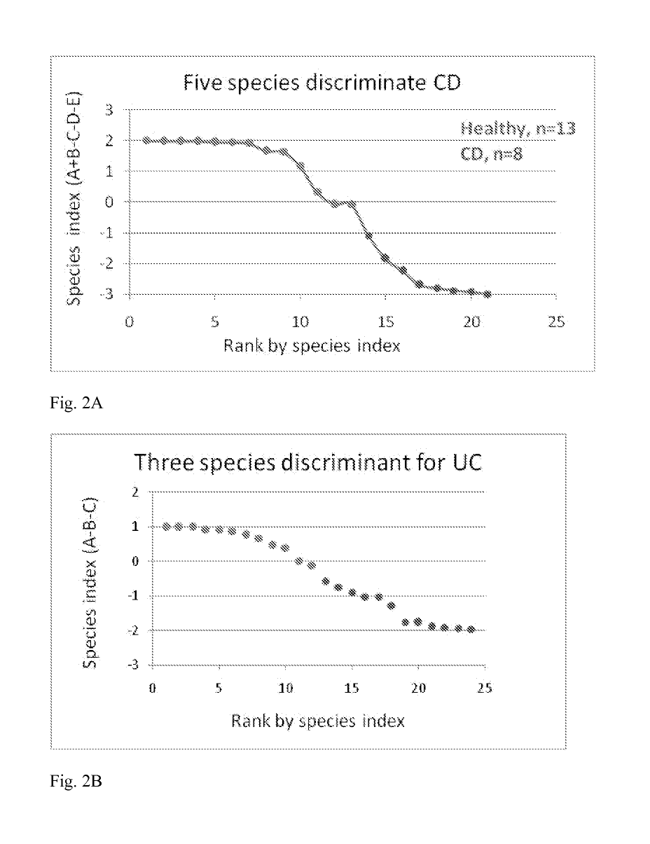

CD-related and UC-related species. The CD-related genes and the UC-related genes were allocated to species, using the taxonomic assignments attributed to the genes in the 3.3 million catalog (Qin et al., Nature, 2010, in press, doi:10.1038/nature08821). It was found that 68% of the CD-related genes, but only 32.8% of all genes, were from firmicutes. On the other hand, the frequency of bacteroidetes was 22% for CD-related genes and 18.4% for all the genes of the microbiome. Likewise, 70% of the UC-related genes were from firmicutes, and only 15% were from bacteroidetes. Therefore, inflammatory-bowel diseases, such as Crohn's disease and ulcerative disease, are associated to changes in firmicutes. The species were first identified by the number of genes assigned to them amongst the CD-related genes and UC-related genes. Then other genes from the same species were pulled out of the catalog and the presence of 50 representative genes for each species assessed in different individuals (this compared very favorably with the use of a single 16S gene, which is currently done to identify a species). The species was considered present if at least half of the marker genes were found in an individual. The significance of the distribution between the healthy and the patients was estimated by the comparison with the all cohort distribution (13 to 8 for Crohn's disease; 12 to 12 for ulcerative colitis) using the Chi2 test. For Crohn disease, Faecalibacterium prausnitzii and Roseburia inulinivorans were associated with the healthy population (p=2.4×10⁻² and p=9.3×10⁻³, respectively), i.e. they tended to be absent from the patients. On the other hand, Clostridium boltae, Clostridium ramosum and Ruminococcus gnavus were associated with the patient cohort (p=4×10⁻³, p=1.8×10⁻³ and p=6.4×10⁻³). On the basis of the identification of species, it was demonstrated that the linear combination of these 5 species fully predicts the Crohn disease phenotype (FIG. 2A). Healthy individuals and patients are shown as blue and red dots, respectively. The species presence (the ordinate) corresponds to the sum of the genes the of “good species” (anti-associated with Crohn's disease) minus the genes of the “bad species” (associated with Crohn's disease).

The individuals are ranked by the species presence (the abscissa). If an individual has excess of the “good species” genes, he or she will be on the top of the rank and tend to be healthy, while if there is an excess of “bad species” genes, he or she will be at the right and tend to be sick. For ulcerative colitis, Akkermansia muciniphila was associated with a healthy phenotype, whereas Bacteroides capillosus and Clostridium leptum were associated with the patient population. As shown in FIG. 2B, a linear combination of the 3 species predicts the ulcerative colitis phenotype.

Lengthy table referenced here
US20130045874A1-20130221-T00001
Please refer to the end of the specification for access instructions.

Lengthy table referenced here
US20130045874A1-20130221-T00002
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20130045874A1). An electronic copy of the table will also be available from the USPTO upon request and payment of the fee set forth in 37 CFR 1.19(b)(3).

1. A method for diagnosing an inflammatory bowel disease in an individual, comprising determining whether at least one gene from Table 1, Table 2, or both is absent from the individual's gut microbiome.

2. The method of claim 1 wherein the inflammatory bowel disease is Crohn's disease or ulcerative colitis.

3. The method of claim 1 wherein at least 50%, 75% or 90% of the genes of Table 1, Table 2, or both are absent from the said individual's gut microbiome.

4. The method of claim 1, comprising obtaining microbial DNA from faeces of the individual.

5. A method for monitoring the efficacy of a treatment for an inflammatory bowel Disease in a patient comprising first determining whether at least one gene from Table 1, Table 2, or both is absent from the patient's microbiome, administering the treatment, and determining if the at least one gene is present in the patient's gut microbiome after treatment.

6. The method of claim 5 wherein the inflammatory bowel disease is Croh's disease or ulcerative colitis.

7. The method of claim 5 wherein at least 50%, 75% or 90% of the genes of Table 1, Table 2, or both are absent from the patient's gut microbiome.

8. The method of claim 5 comprising at least one step of obtaining microbial DNA from faeces of the said patient.

9-10. (canceled)

11. The method of claim 1 wherein at least 90% of the genes of Table 1, Table 2, or both are absent from the individual's gut microbiome.

12. The method of claim 1 which comprises:
obtaining a sample from the individual;
extracting microbial DNA from the sample;
measuring the level of at least 10%, 25%, 50%, 75%, 90%, 95%, 97.5% or 99% of the genes of Table 1, Table 2 or both in the sample; and
determining whether at least one gene from Table 1, Table 2 or both is absent from the individual's gut microbiome.

13. The method of claim 12 wherein said measuring of the level of genes is determined by sequencing, quantitative PCR, Southern hybridization, or microarray.

14. The method of claim 12 wherein the gene is absent from the individual's gut microbiome when its number of copies in the microbiome is under a certain threshold value.

15. The method of claim 12 wherein inflammatory bowel disease is diagnosed in the individual when at least 50%, 75% or 90% of the genes of Table 1, Table 2 or both are absent from the individual's gut microbiome.

16. The method of claim 5 which comprises:
obtaining a sample from the individual;
extracting microbial DNA from the sample;
measuring the level of at least 10%, 25%, 50%, 75%, 90%, 95%, 97.5% or 99% of the genes of Table 1, Table 2 or both in the sample;
determining whether at least one gene from Table 1, Table 2 or both is absent from the individual's gut microbiome;
administering the treatment for an inflammatory bowel disease;
obtaining a subsequent sample from the individual;
extracting microbial DNA from the subsequent sample;
determining if said at least one gene from Table 1, Table 2 or both is present in the individual's gut microbiome after treatment.

17. A microarray comprising probes hybridizing to at least 10% of the genes of Table 1, Table 2 or both.

18. The microarray of claim 17 comprising probes hybridizing to at least 50% of the genes of Table 1, Table 2 or both.

19. The microarray of claim 17 comprising probes hybridizing to at least 95% of the genes of Table 1, Table 2 or both.

20. The microarray of claim 17 comprising probes hybridizing to at least 99% of the genes of Table 1, Table 2 or both.

21. A kit for diagnosing an inflammatory bowel disease comprising a microarray of claim 17 or amplification primers specific for at least 10% of the genes of Table 1, Table 2 or both.

22. A kit for diagnosing an inflammatory bowel disease comprising a microarray of claim 20 or amplification primers specific for at least 99% of the genes of Table 1, Table 2 or both.
